(19) United States
(12) Patent Application Publication
(10) Pub. No.: US 2011/0294764 A1
(43) Pub. Date: Dec. 1, 2011

(54) 2-METHYLENE-19,26-NOR-(20S)-1alpha-HYDROXYVITAMIN D3

(76) Inventors: Hector F. DeLuca, Deerfield, WI (US); Pawel Grzywacz, Madison, WI (US); Lori A. Plum, Arena, WI (US); Margaret Clagett-Dame, Deerfield, WI (US)

(21) Appl. No.: 12/955,567

(22) Filed: Nov. 29, 2010

Related U.S. Application Data
(60) Provisional application No. 61/264,990, filed on Nov. 30, 2009.

(51) Int. Cl. A61K 31/593; C07F 7/18; A61P 17/06; A61P 35/02; A61P 35/00; A61P 25/00; A61P 19/04; A61P 3/10; A61P 37/06; A61P 29/00; A61P 11/06; A61P 1/00; A61P 17/00; A61Q 19/08; A61P 13/12; A61P 19/10; A61P 1/04; C07C 35/21

(52) U.S. Cl. 514/167; 568/819; 556/444

(57) ABSTRACT

Compounds of formula I are provided where X¹ and X² are independently selected from H or hydroxy protecting groups. Such compounds may be used in preparing pharmaceutical compositions and are useful in treating a variety of biological conditions.

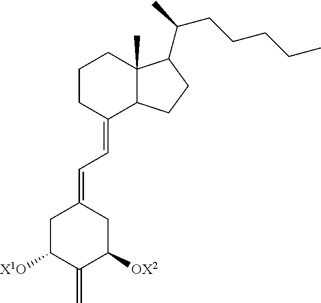

### CROSS-REFERENCE TO RELATED APPLICATIONS

This application claims priority to U.S. Provisional Patent Application No. 61/264,990 filed Nov. 30, 2009, the entire disclosure of which is hereby incorporated by reference in its entirety.

### FIELD

This present technology relates to vitamin D compounds, and more particularly to 2-methylene-19,26-nor-(20S)-1α-hydroxyvitamin D₃ and derivatives thereof, and to pharmaceutical formulations that include this compound. The present technology also relates to the use of 2-methylene-19,26-nor-(20S)-1α-hydroxyvitamin D₃ in the treatment of various diseases and in the preparation of medicaments for use in treating various diseases.

### BACKGROUND

The natural hormone, 1α,25-dihydroxyvitamin D₃ (also referred to as 1α,25-dihydroxycholecalciferol and calcitriol) and its analog in the ergosterol series, i.e. 1α,25-dihydroxyvitamin D₂, are known to be highly potent regulators of calcium homeostasis in animals and humans, and their activity in cellular differentiation has also been established, Ostrem et al., Proc. Natl. Acad. Sci. USA, 84, 2610 (1987). Many structural analogs of these metabolites have been prepared and tested, including 1α-hydroxyvitamin D₃, 1α-hydroxyvitamin D₂, various side chain homologated vitamins, and fluorinated analogs. Some of these compounds exhibit an interesting separation of activities in cell differentiation and calcium regulation. This difference in activity may be useful in the treatment of a variety of diseases such as renal osteodystrophy, vitamin D-resistant rickets, osteoporosis, psoriasis, and certain malignancies. The structure of 1α,25-dihydroxyvitamin D₃ and the numbering system used to denote the carbon atoms in this compound are shown below.

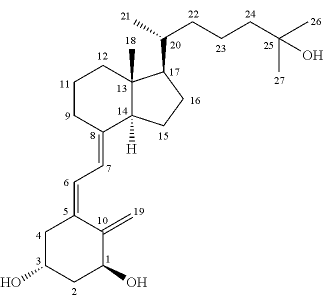

1α,25-Dihydroxyvitamin D₃=1α,25-Dihydroxycholecalciferol=Calcitriol

### SUMMARY

The present technology provides 2-methylene-19,26-nor-(20S)-1α-hydroxyvitamin D3 and related compounds, pharmaceutical formulations that include 2-methylene-19,26-nor-(20S)-1α-hydroxyvitamin D₃, methods of treating various disease states using this compound, and the use of this compound in the preparation of medicaments for treating various disease states.

Therefore, in one aspect, the present technology provides a compound having the formula I shown below:

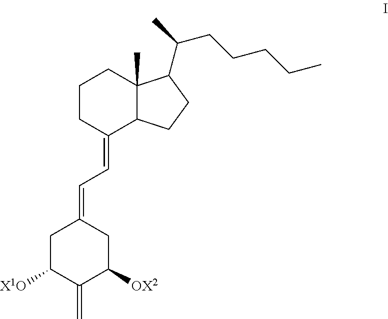

where X¹ and X² may be the same or different and are independently selected from H or hydroxy-protecting groups. In some embodiments, X¹ and X² are both hydroxy protecting groups such as silyl groups. In some such embodiments, X¹ and X² are both t-butyldimethylsilyl groups. In other embodiments, X¹ and X² are both H such that the compound is 2-methylene-19,26-nor-(20S)-1α-hydroxyvitamin D₃ having the formula IA as shown below:

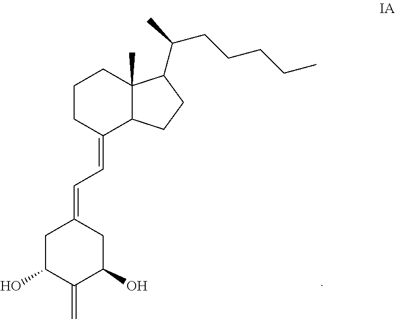

In some such embodiments, the compound of formula IA is a compound of formula IB and has the structure shown below:

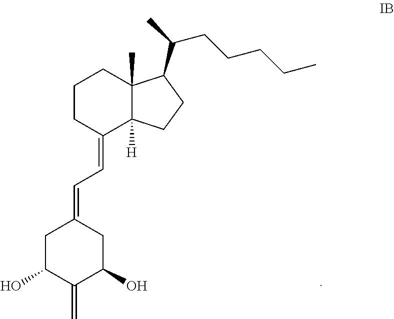

Compounds of the present technology show a highly advantageous pattern of biological activity, including strong binding to the vitamin D receptor, strong cell differentiation activity, yet low to very low calcemic activity. Thus the present compounds may be used in methods of treating a subject suffering from certain biological conditions and for the preparation of medicaments for treating such conditions. The methods include administering an effective amount of a compound of the present technology to the subject, wherein the biological condition is selected from psoriasis; leukemia; colon cancer; breast cancer; prostate cancer; multiple sclerosis; lupus; diabetes mellitus; host versus graft reaction; rejection of organ transplants; an inflammatory disease selected from rheumatoid arthritis, asthma, or inflammatory bowel diseases; a skin condition selected from wrinkles, lack of adequate skin firmness, lack of adequate dermal hydration, or insufficient sebum secretion; renal osteodystrophy; or osteoporosis.

A compound of the present technology may be present in a composition to treat the above-noted diseases and disorders in an effective amount and optionally including a pharmaceutically acceptable carrier. In some embodiments, the amount of compound includes from about 0.01 μg per gram to about 1 mg per gram of the composition, preferably from about 0.1 μg per gram to about 500 μg per gram of the composition, and may be administered topically, transdermally, orally, or parenterally in dosages of from about 0.01 μg per day to about 1 mg per day, preferably from about 0.1 μg per day to about 500 μg per day.

Further features and advantages of the present technology will be apparent from the following detailed description and drawings.

### BRIEF DESCRIPTION OF THE DRAWINGS

FIGS. 1-5 illustrate various biological activities of 2-methylene-19,26-nor-(20S)-1α-hydroxyvitamin D3 (referred to as “26N” in the Figures) compared with those of the native hormone 1α,25-dihydroxyvitamin D₃ (referred to as “1,25(OH)₂D₃” in the Figures).

FIG. 1 shows a graph of competitive binding to the nuclear hormone receptor between 26N and the native hormone, 1,25(OH)₂D₃. 26N binds to the nuclear vitamin D receptor with the same affinity as 1,25(OH)₂D₃.

FIG. 2 is a graph comparing the percent HL-60 cell differentiation as a function of the concentration of 26N with that of 1,25(OH)₂D₃, 26N has the same potency as 1,25(OH)₂D₃ in causing the differentiation of HL-60 cells into monocytes.

FIG. 3 is a graph comparing the in vitro transcription activity of 26N with that of 1,25(OH)₂D₃. 26 N is about one log less potent than 1,25(OH)₂D₃ in increasing transcription of the 24-hydroxylase gene.

FIGS. 4A and 4B are bar graphs comparing the bone calcium mobilization activity of 26N with that of 1,25(OH)₂D₃ in rat. 26N is approximately 30 times less potent than 1,25(OH)₂D₃ in releasing bone calcium stores.

FIGS. 5A and 5B are bar graphs comparing the intestinal calcium transport activity of 26N with that of 1,25(OH)₂D₃. 26N is at least 10 times less potent than 1,25(OH)₂D₃ in promoting active calcium transport in the rat gut.

### DETAILED DESCRIPTION

2-Methylene-19,26-nor-(20S)-1α-hydroxyvitamin D₃ was synthesized, and tested, and found to be useful in treating a variety of biological conditions as described herein. Structurally, this compound has the formula IA as shown below:

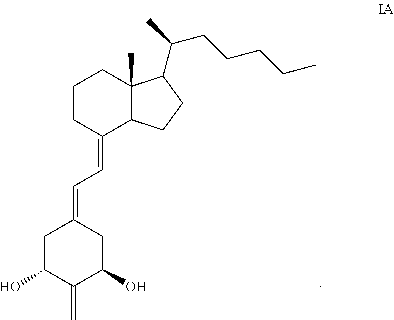

Preparation of 2-methylene-19,26-nor-(20S)-1α-hydroxyvitamin D₃ can be accomplished by condensing an appropriate bicyclic Windaus-Grundmann type ketone (II) with the allylic phosphine oxide III followed by deprotection (removal of the Y₁ and Y₂ groups).

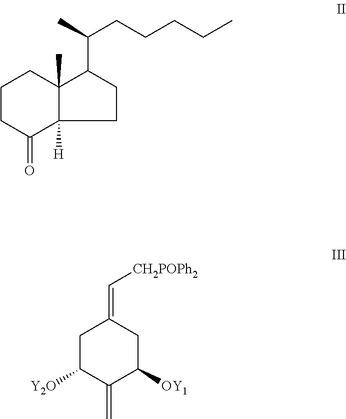

In phosphine oxide III, Y₁ and Y₂ are hydroxy-protecting groups such as silyl protecting groups. The t-butyldimethylsilyl (TBDMS) group is an example of a particularly useful hydroxy-protecting group. The process described above represents an application of the convergent synthesis concept, which has been applied effectively to the preparation of numerous vitamin D compounds (see Lythgoe et al., J. Chem. Soc. Perkin Trans. I, 590 (1978); Lythgoe, Chem. Soc. Rev. 9, 449 (1983); Toh et al., J. Org. Chem. 48, 1414 (1983); Baggiolini et al., J. Org. Chem. 51, 3098 (1986); Sardina et al., J. Org. Chem. 51, 1264 (1986); J. Org. Chem. 51, 1269 (1986); DeLuca et al., U.S. Pat. No. 5,086,191; DeLuca et al., U.S. Pat. No. 5,536,713; and DeLuca et al., U.S. Pat. No. 5,843,928 all of which are hereby incorporated by reference in their entirety and for all purposes as if fully set forth herein).

Phosphine oxide III is a convenient reagent that may be prepared according to the procedures described by Sicinski et al., J. Med. Chem., 41, 4662 (1998), DeLuca et al., U.S. Pat. No. 5,843,928; Penman et al., Tetrahedron Lett. 32, 7663 (1991); and DeLuca et al., U.S. Pat. No. 5,086,191. Scheme 1 shows the general procedure for synthesizing phosphine oxide III as outlined in U.S. Pat. No. 5,843,928 which is hereby incorporated by reference in its entirety as if fully set forth herein.

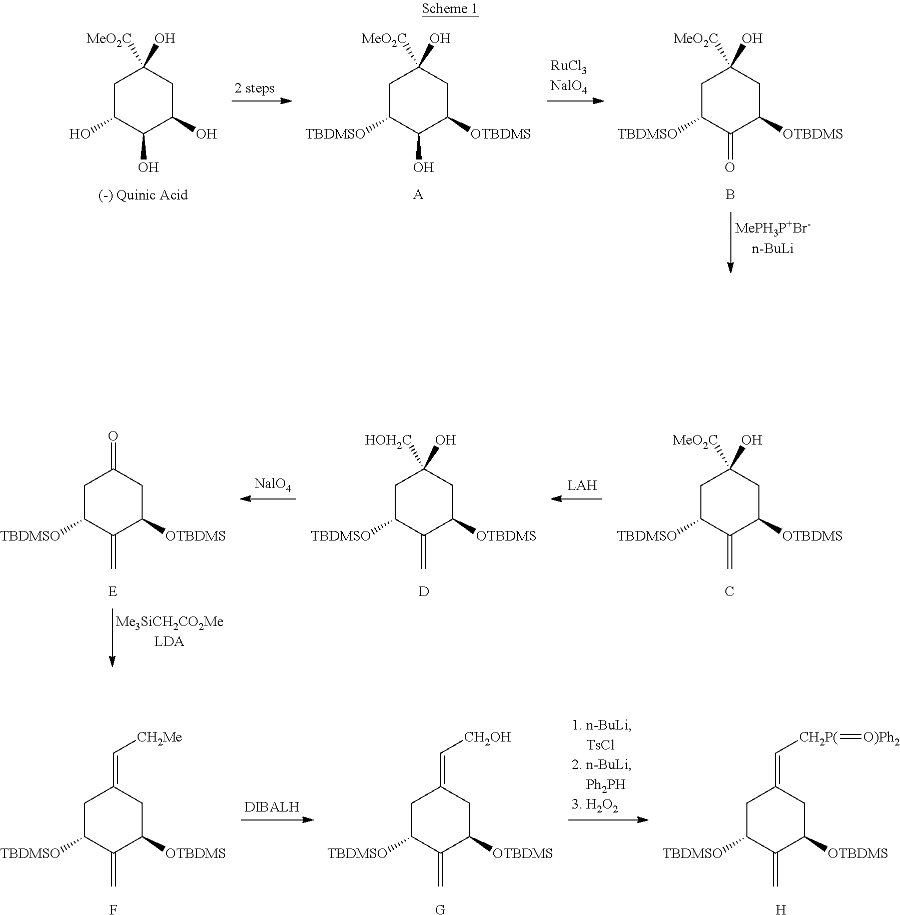

Hydraindanones of structure can prepared by slight modification of known methods as will be readily apparent to one of skill in the art and described herein. Specific examples of methods used to synthesize bicyclic ketones for vitamin D analogs are those described in Mincione et al., Synth. Commun 19, 723, (1989); and Peterson et al., J. Org. Chem. 51, 1948, (1986). An overall process for synthesizing 2-alkylidene-19-nor-vitamin D compounds is illustrated and described in U.S. Pat. No. 5,843,928 which is hereby incorporated by reference in its entirety and for all purposes as if fully set forth herein. Details of preparing hydrinanone II are found in Scheme 2 and the Examples herein.

As used herein, the term “hydroxy-protecting group” signifies any group commonly used for the temporary protection of the hydroxy (—OH) functional group, such as, but not limited to, alkoxycarbonyl, acyl, alkylsilyl or alkylarylsilyl groups (hereinafter referred to simply as “silyl” groups), and alkoxyalkyl groups. The term “alkyl.” refers to straight or branched chain saturated hydrocarbon groups of 1 to 6 carbons and also includes cycli. Alkoxycarbonyl protecting groups are alkyl-O—CO— groups such as methoxycarbonyl, ethoxycarbonyl, propoxycarbonyl, isopropoxycarbonyl, butoxycarbonyl, isobutoxycarbonyl, tert-butoxycarbonyl, benzyloxycarbonyl or allyloxycarbonyl. The term “acyl” signifies alkanoyl group of 1 to 6 carbons, in all of its isomeric forms, or a carboxyalkanoyl group of 1 to 6 carbons, such as an oxalyl, malonyl, succinyl, glutaryl group, or an aromatic acyl group such as benzoyl, or a halo, nitro or alkyl substituted benzoyl group. Alkoxyalkyl protecting groups are groups such as methoxymethyl, ethoxymethyl, methoxyethoxymethyl, or tetrahydrofuranyl and tetrahydropyranyl. Preferred silyl-protecting groups are trimethylsilyl, triethylsilyl, t-butyldimethylsilyl, dibutylmethylsilyl, diphenylmethylsilyl, phenyldimethylsilyl, diphenyl-t-butylsilyl and analogous alkylated silyl radicals. The term “aryl” specifies a phenyl-, or an alkyl-, nitro- or halo-substituted phenyl group. An extensive list of protecting groups for the hydroxy functionality may be found in Protective Groups in Organic Synthesis, Greene, T. W.; Wuts, P. G. M., John Wiley & Sons, New York, N.Y., (3rd Edition, 1999) which can be added or removed using the procedures set forth therein and which is hereby incorporated by reference in its entirety and for all purposes as if fully set forth herein.

A “protected hydroxy” group is a hydroxy group derivatized or protected by any of the above groups commonly used for the temporary or permanent protection of hydroxy functional groups, e.g., the alkoxyalkyl, acyl or alkoxycarbonyl groups, as previously defined.

The above compound exhibits a desired, and highly advantageous, pattern of biological activity. This compound is characterized by relatively high binding to vitamin D receptors, but very low intestinal calcium transport activity, as compared to that of 1α,25-dihydroxyvitamin D₃, and has low ability to mobilize calcium from bone, as compared to 1α,25-dihydroxyvitamin D₃. Hence, this compound can be characterized as having little, if any, calcemic activity at the dosages that 1α,25-dihydroxyvitamin D₃ displays significant calcemic activity. Thus, it may be useful as a therapy for suppression of secondary hyperparathyroidism of renal osteodystrophy.

The compound of the present technology is also especially suited for treatment and prophylaxis of human disorders which are characterized by an imbalance in the immune system, e.g. in autoimmune diseases, including multiple sclerosis, lupus; diabetes mellitus, host versus graft reaction, and rejection of organ transplants; and additionally for the treatment of inflammatory diseases, such as rheumatoid arthritis, asthma, and inflammatory bowel diseases such as celiac disease, ulcerative colitis and Crohn's disease. Acne, alopecia and hypertension are other conditions which may be treated with the compound of the present technology.

The above compound is also characterized by relatively high cell differentiation activity. Thus, this compound also provides a therapeutic agent for the treatment of psoriasis, or as an anti-cancer agent, especially against leukemia, colon cancer, breast cancer and prostate cancer. In addition, due to its relatively high cell differentiation activity, this compound provides a therapeutic agent for the treatment of various skin conditions including wrinkles, lack of adequate dermal hydration, i.e. dry skin, tack of adequate skin firmness, i.e. slack skin, and insufficient sebum secretion. Use of this compound thus not only results in moisturizing of skin but also improves the barrier function of skin.

The compounds of the present technology may be used to prepare pharmaceutical formulations or medicaments that include a compound of the present technology in combination with a pharmaceutically acceptable carrier. Such pharmaceutical formulations and medicaments may be used to treat various biological disorders such as those described herein. Methods for treating such disorders typically include administering an effective amount of the compound or an appropriate amount of a pharmaceutical formulation or a medicament that includes the compound to a subject suffering from the biological disorder. In some embodiments, the subject is a mammal. In some such embodiments, the mammal is selected from a rodent, a primate, a bovine, an equine, a canine, a feline, an ursine, a porcine, a rabbit, or a guinea pig. In some such embodiments, the mammal is a rat or is a mouse. In some embodiments, the subject is a primate such as, in some embodiments, a human.

For treatment purposes, the compounds defined by formula I, formula IA; and formula IB may be formulated for pharmaceutical applications as a solution in innocuous solvents, or as an emulsion, suspension or dispersion in suitable solvents or carriers, or as pills, tablets or capsules, together with solid carriers, according to conventional methods known in the art. Any such formulations may also contain other pharmaceutically acceptable and non-toxic excipients such as stabilizers, anti-oxidants, binders, coloring agents or emulsifying or taste-modifying agents. Pharmaceutically acceptable excipients and carriers are generally known to those skilled in the art and are thus included in the present technology. Such excipients and carriers are described, for example, in “Remingtons Pharmaceutical Sciences” Mack Pub. Co., New Jersey (1991), which is hereby incorporated by reference in its entirety and for all purposes as if fully set forth herein.

The compounds may be administered orally, topically, parenterally, or transdermally. The compounds are advantageously administered by injection or by intravenous infusion or suitable sterile solutions, or in the form of liquid or solid doses via the alimentary canal, or in the form of creams, ointments, patches, or similar vehicles suitable for transdermal applications. In some embodiments, doses of from 0.001 μg to about 1 mg per day of the compound are appropriate for treatment purposes. In some such embodiments an appropriate and effective dose may range from 0.01 μg to 1 mg per day of the compound. In other such embodiments an appropriate and effective dose may range from 0.1 μg to 500 μg per day of the compound. Such doses will be adjusted according to the type of disease or condition to be treated, the severity of the disease or condition, and the response of the subject as is well understood in the art. The compound may be suitably administered alone, or together with another active vitamin D compound.

Compositions for use in the present technology include an effective amount of 2-methylene-19,26-nor-(20S)-1α-hydroxyvitamin D3 as the active ingredient, and a suitable carrier. An effective amount of the compound for use in accordance with some embodiments of the present technology will generally be a dosage amount such as those described herein, and may be administered topically, transdermally, orally, nasally, rectally, or parenterally.

The compound of formula IA and formula IB may be advantageously administered in amounts sufficient to effect the differentiation of promyelocytes to normal macrophages. Dosages as described above are suitable, it being understood that the amounts given are to be adjusted in accordance with the severity of the disease, and the condition and response of the subject as is well understood in the art.

The compound may be formulated as creams, lotions, ointments, aerosols, suppositories, topical patches, pills, capsules or tablets, or in liquid form as solutions, emulsions, dispersions, or suspensions in pharmaceutically innocuous and acceptable solvent or oils, and such preparations may contain, in addition, other pharmaceutically innocuous or beneficial components, such as stabilizers, antioxidants, emulsifiers, coloring agents, binders or taste-modifying agents.

The formulations of the present technology comprise an active ingredient in association with a pharmaceutically acceptable carrier therefore and optionally other therapeutic ingredients. The carrier must be “acceptable” in the sense of being compatible with the other ingredients of the formulations and not deleterious to the recipient thereof.

Formulations of the present technology suitable for oral administration may be in the form of discrete units as capsules, sachets, tablets or lozenges, each containing a predetermined amount of the active ingredient; in the form of a powder or granules; in the form of a solution or a suspension in an aqueous liquid or non-aqueous liquid; or in the form of an oil-in-water emulsion or a water-in-oil emulsion.

Formulations for rectal administration may be in the form of a suppository incorporating the active ingredient and carrier such as cocoa butter, or in the form of an enema.

Formulations suitable for parenteral administration conveniently comprise a sterile oily or aqueous preparation of the active ingredient which is preferably isotonic with the blood of the recipient.

Formulations suitable for topical administration include liquid or semi-liquid preparations such as liniments, lotions, applicants, oil-in-water or water-in-oil emulsions such as creams, ointments or pastes; or solutions or suspensions such as drops; or as sprays.

For nasal administration, inhalation of powder, self-propelling or spray formulations, dispensed with a spray can, a nebulizer or an atomizer can be used. The formulations, when dispensed, preferably have a particle size in the range of 10 to 100 microns.

The formulations may conveniently be presented in dosage unit form and may be prepared by any of the methods well known in the art of pharmacy. By the term “dosage unit” is meant a unitary, i.e., a single dose which is capable of being administered to a patient as a physically and chemically stable unit dose comprising either the active ingredient as such or a mixture of it with solid or liquid pharmaceutical diluents or carriers.

All references cited herein are specifically incorporated by reference in their entireties and for all purposes as if fully set forth herein.

### EXAMPLES

### Synthesis of 2-methylene-19,26-nor-(20S)-1α-hydroxyvitamin D3

Compounds of formula I, formula IA, and formula IIB were prepared using the methods shown in Schemes 1-3. As shown in Scheme 2, compound 1 is obtained by ozonolysis of vitamin D₂ as described by Grywacz et (Arch. Biochem. Biophys. 460, 274-284, 2007), followed by reduction with borohydride. Treatment of the dialcohol 1 with benzoyl chloride and DMAP in pyridine followed by KOH in ethanol provides the benzoyl compound 2. Compound 2 is oxidized with sulfur trioxide pyridine complex in the presence of TEA in dichloromethane and DMSO to yield compound 3. Compound 3 was epimerized at position 20 by treatment with tetrabutylammonium hydroxide and then reduced with sodium borohydride to give compound 4. The latter compound was converted to the tosylate by treatment with tosyl chloride, TEA and DMAP in dichloromethane to yield compound 5. Reaction of tosylate 5 with butyl magnesium chloride in the presence of Li₂CuCl₄ provided, after workup using 1M sulfuric acid, the free alcohol 7. Compound 7 was oxidized to compound 8 using tetrapropylammonium perruthenate in the presence of 4-methylmorpholine oxide.

The Ring-A phosphine oxide compound 9 was synthesized as shown in Scheme 1 and as previously described. As shown in Scheme 3, compound 8 was coupled with the A-ring phosphonium salt using phenyl lithium as set forth in the above-referenced patent documents to produce compound 10, the t-butyldimethylsityl (TBDMS) protected vitamin derivative. Removal of the protecting groups from compound 10 with hydrofluoric acid (HF) in acetonitrile (MeCN) and in tetrahydrofuran (THF) provided the desired product compound 11 (compound of Formula IA) and is detected using TLC using 50% ethyl acetate in hexane. This product was fully characterized as described below.

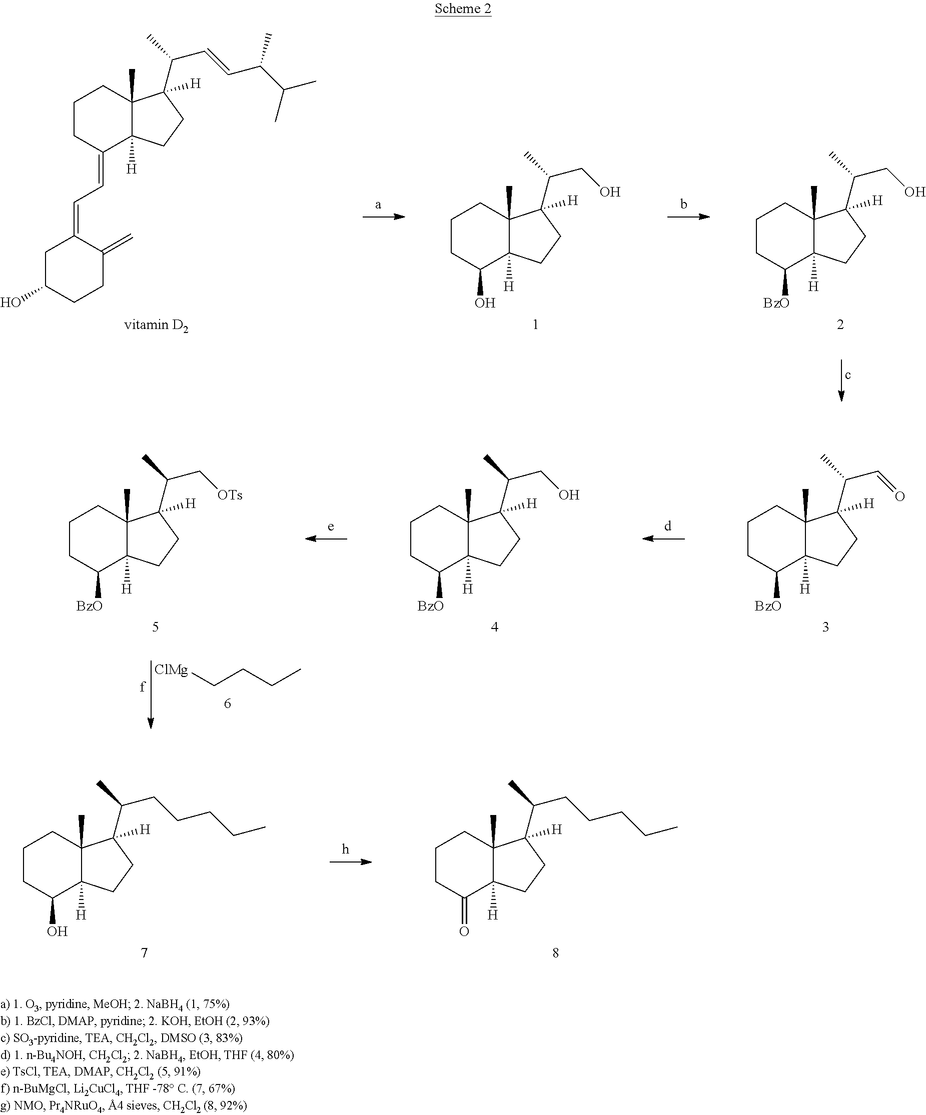

### Preparation of (8S,20S)-Des-A,B-20-(hydroxymethyl)pregnan-8-ol (1)

Ozone was passed through a solution of vitamin D₂ (3 g, 7.6 mmol) in methanol (250 mL) and pyridine (2.44 g, 2.5 mL, 31 mmol) for 50 min at −78° C. The reaction mixture was then flushed with an oxygen for 15 min to remove the residual ozone and the solution was treated with NaBH₄ (0.75 g, 20 mmol). After 20 min the second portion of NaBH₄ (0.75 g, 20 mmol) was added, and the mixture was allowed to warm to room temperature. The third portion of NaBH₄ (0.75 g, 20 mmol) was then added and the reaction mixture was stirred for 18 h. The reaction was quenched with water (40 mL), and the solution was concentrated under reduced pressure. The residue was extracted with ethyl acetate and the combined organic phases were washed with 1 M aq. HCl, saturated aq. NaHCO₃, dried (Na₂SO₄) and concentrated under reduced pressure. The residue was chromatographed on silica gel with hexane/ethyl acetate (75:25) to give the diol 1 (1.21 g, 75% yield) as white crystals: m.p. 106-108° C.; [α]_{D} +30.2° (c 1.46, CHCl₃); ¹H NMR (400 MHz, CDCl₃) δ 4.08 (1H, d, J=2.0 Hz, 8α-H), 3.63 (1H, dd, J=10.5, 3.1 Hz, 22-H), 3.38 (1H, dd, J=10.5, 6.8 Hz, 22-H), 1.99 (1H, br.d, J=13.2 Hz), 1.03 (3H, d, J=6.6 Hz, 21-H₃), 0.956 (3H, s, 18-H₃); ¹³C NMR (100 MHz) δ 69.16 (d, C-8), 67.74 (t, C-22), 52.90 (d), 52.33 (d), 41.83 (s, C-13), 40.19 (t), 38.20 (d), 33.53 (t), 26.62 (t), 22.54 (t), 17.36 (t), 16.59 (q, C-21), 13.54 (q, C-18); MS (EI) m/z 212 (2, M⁺), 194 (34, M⁺-H₂O), 179 (33, M⁺-H₂O—CH₃), 163 (18, M⁺-CH₂OH—H₂O), 135 (36), 125 (54), 111 (100), 95 (63), 81 (67); exact mass calculated for C₁₃H₂₂O (M⁺-H₂O) 194.1671, found 194.1665.

### Preparation of (8S,20)-Des-A,B-8-benzoyloxy-20-(hydroxymethyl)pregnane (2)

Benzoyl chloride (2.4 g, 2 mL, 17 mmol) was added to a solution of the diol 1 (1.2 g, 5.7 mmol) and DMAP (30 mg, 0.2 mmol) in anhydrous pyridine (20 at 0° C. The reaction mixture was stirred at 4° C. for 24 h, diluted with methylene chloride (100 mL), washed with 5% aq. HCl, water, saturated aq. NaHCO₃; dried (Na₂SO₄) and concentrated under reduced pressure. The residue (3.39 g) was treated with a solution of KOH (1 g, 15.5 mmol) in anhydrous ethanol (30 mL) at room temperature. After stirring of the reaction mixture for 3 h, ice and 5% aq. HCl were added until pH=6. The solution was extracted with ethyl acetate (3×50 mL) and the combined organic phases were washed with saturated aq. NaHCO₃, dried (Na₂SO₄) and concentrated under reduced pressure. The residue was chromatographed on silica gel with hexane/ethyl acetate (75:25) to give the alcohol 2 (1.67 g, 93% yield) as a colorless oil: [α]_{D} +56.0 (c 0.48, CHCl₃); ¹H NMR (400 MHz, CDCl₃+TMS) δ 8.08-8.02 (2H, m, o-H_{Bz}), 7.59-7.53 (1H, m, p-H_{Bz}), 7.50-7.40 (2H, m, m-H_{Bz}), 5.42 (1H, d, J=2.4 Hz, 8α-H), 3.65 (1H, dd, J=10.5, 3.2 Hz, 22-H), 3.39 (1H, dd, J=10.5, 6.8 Hz, 22-H), 1.08 (3H, d, J=5.3 Hz, 21-H₃), 1.07 (3H, s, 18-H₃); ¹³C NMR (125 MHz) δ 166.70 (s, C═O), 132.93 (d, p-C_{Bz}), 130.04 (s, i-C_{Bz}), 129.75 (d, o-C_{Bz}), 128.57 (d, m-C_{Bz}), 72.27 (d, C-8), 67.95 (t, C-22), 52.96 (d), 51.60 (d), 42.15 (s, C-13), 39.98 (t), 38.61 (d), 30.73 (t), 26.81 (t), 22.91 (t), 18.20 (t), 16.87 (q, C-21), 13.81 (q, C-18); MS (EI) m/z 316 (5, M⁺), 301 (3, M⁺-Me), 299 (1, M⁺-OH), 298 (2, M⁺-H₂O), 285 (10, M⁺CH₂OH), 257 (6), 230 (9), 194 (80), 135 (84), 105 (100); exact mass calculated for C₂₀H₂₈O₃ (M⁺) 316.2038, found 316.2019.

### Preparation of (8S,20S)-Des-A,B-8-benzoyloxy-20-formylpregnane (3)

Sulfur trioxide pyridine complex (1.94 g, 12.2 mmol) was added to a solution of the alcohol 2 (6.40 mg, 2.03 mmol), triethylamine (1.41 mL, 1.02 g, 10.1 mmol) in anhydrous methylene chloride (10 mL) and anhydrous DMSO (2 mL) at 0° C. The reaction mixture was stirred under argon at 0° C. for 1 h and then concentrated. The residue was diluted with ethyl acetate, washed with brine, dried (Na₂SO₄) and concentrated. The residue was purified by column chromatography on silica gel with hexane/ethyl acetate (95:5) to give the aldehyde 3 (529 mg, 83% yield) as an oil: ¹H NMR (400 MHz, CDCl₃+TMS) δ 9.60 (1H, d, J=3.1 Hz, CHO), 8.05 (2H, m, o-H_{Bz}), 7.57 (1H, m, p-H_{Bz}), 7.45 (2H, m, m-H_{Bz}), 5.44 (1H, s, 8α-H), 2.39 (1H, m, 20-H), 2.03 (2H, dm, J=11.5 Hz), 1.15 (3H, d, J=6.9 Hz, 21-H₃), 1.10 (3H, s, 18-H₃); ¹³C NMR (100 MHz) δ 204.78 (d, CHO), 166.70 (s, C═O), 132.78 (d, p-Bz), 130.69 (s, i-Bz), 129.50 (d, o-Bz), 128.38, (d, m-Bz), 71.66 (d, C-8), 51.30 (d), 50.95 (d), 49.20 (d), 42.38 (s, C-13), 39.62 (t), 30.47 (t), 25.99 (1), 22.92 (t), 17.92 (t), 13.90 (q), 13.35 (q); MS (EI) m/z 314 (1, M⁺), 299 (0.5, M⁺-Me), 286 (1, M⁺-CO), 285 (5, M⁺-CHO), 257 (1, M⁺-C₃H₅O), 209 (10, M⁺-PhCO), 192 (38), 134 (60), 105 (100), 77 (50); exact mass calculated for C₂₀H₂₆O₃ (M⁺) 314.1882, found 314.1887.

### Preparation of (8S,20R)-Des-A,B-8-benzoyloxy-20-(hydroxymethyl)pregnane (4)

The aldehyde 3 (364 mg, 1.12 mmol) was dissolved in methylene chloride (15 mL) and a 40% aq. n-Bu₄NOH solution (1.47 mL, 1.45 g, 2.24 mmol) was added. The resulting mixture was stirred under argon at room temperature for 16 h, diluted with methylene chloride (20 mL), washed with water, dried (Na₂SO₄) and concentrated under reduced pressure. A residue was chromatographed on silica gel with hexane/ethyl acetate (95:5) to afford a mixture of aldehyde 3 and its 20-epimer (292 mg, 80% yield) in ca. 1:2 ratio (by ¹H NMR).

This mixture of aldehydes (292 mg, 0.9 mmol) was dissolved in THF (5 mL) and NaBH₄ (64 mg, 1.7 mmol) was added, followed by a dropwise addition of ethanol (5 mL). The reaction mixture was stirred at room temperature for 30 min and it was quenched with a saturated aq. NH₄Cl solution. The mixture was extracted with ether (3×20 mL) and the combined organic phase was washed with water, dried (Na₂SO₄) and concentrated under reduced pressure. The residue was chromatographed on silica gel with hexane/ethyl acetate (96:4→80:20) to give the desired, pure (20R)-alcohol 4 (160 mg, 55% yield) as an oil and a mixture of 4 and its 20-epimer 2 (126 mg, 43% yield) in ca. 1:3 ratio (by ¹H NMR): [α]_{D} +50.1 (c 1.09, CHCl₃); ¹H NMR (400 MHz, CDCl₃+TMS) δ 8.05 (2H, in, o-H_{Bz}), 7.55 (1H, m, p-H_{Bz}), 7.44 (2H, m, m-H_{Bz}), 5.41 (1H, s, 8α-H), 3.77 (1H, dd, J=10.4, 3.3 Hz, 22-H), 3.45 (1H, dd, J=10.4, 7.4 Hz, 22-H), 1.067 (3H, s, 18-H₃), 0.973 (3H, d, J=6.6 Hz, 21-H₃); ¹³C NMR (100 MHz) δ 166.36 (s, C═O), 132.61 (d, p-C_{Bz}), 130.63 (s, i-C_{Bz}), 129.39 (d, o-C_{Bz}), 128.23 (d, m-C_{Bz}), 71.97 (d, C-8), 66.42 (t, C-22), 52.65 (d), 51.38 (d), 41.58 (s, C-13), 39.16 (t), 37.45 (d), 30.38 (t), 26.29 (t), 22.35 (t), 17.89 (t), 16.42 (q, C-21), 13.78 (q, C-18); MS (EI) m/z 316 (16, M⁺), 301 (5, M⁺-Me), 299 (2, M⁺-OH), 298 (3, M⁺-H₂O), 285 (9, M⁺-CH₂OH), 257 (5), 242 (11), 230 (8), 194 (60), 147 (71), 105 (100); exact mass calculated for C₂₀H₂₈O₃ (M⁺) 316.2038, found 316.2050.

### Preparation of (20R)-Des-A,B-8-benzoyloxy-20-[(p-toluenesulfonyl)-oxymethyl]pregnane (5)

To a stirred solution of the alcohol 4 (393 mg, 1.24 mmol), DMAP (10 mg, 0.08 mmol) and Et₃N (0.7 mL, 0.51 g, 5.04 mmol) in anhydrous methylene chloride (10 mL) was added p-toluenesulfonyl chloride (320 mg, 1.68 mmol) at 0° C. The reaction mixture was allowed to warm to room temperature (4 h) and stirring was continued for additional 22 h. Methylene chloride (60 mL) was added and the mixture was washed with a saturated aq. NaHCO₃ solution, dried (Na₂SO₄) and concentrated under reduced pressure. A residue was chromatographed on silica gel with hexane/ethyl acetate (95:5) to afford a tosylate 5 (533 mg, 91% yield) as a colorless oil: [α]_{D} +15.0 (c 0.54, CHCl₃); ¹H NMR (500 MHz, CDCl₃+TMS) δ 8.02 (2H, m, o-H_{Bz}), 7.80 (2H, d, J=8.2 Hz, o-H_{Ts}), 7.55 (1H, m, p-H_{Bz}), 7.44 (2H, m, m-H_{Bz}), 7.35 (2H, d, J=8.2 Hz, m-H_{Ts}), 5.39 (1H, br s, 8α-H), 4.15 (1H, dd, J=9.4, 3.4 Hz, 22-H), 3.83 (1H, dd, J=9.4, 7.1 Hz, 22-H), 2.457 (3H, s, Me_{Ts}), 1.98 (1H, m), 0.978 (3H, s, 18-H₃), 0.898 (3H, d, 6.6 Hz, 21-H₃); ¹³C NMR (125 MHz) δ 166.60 (s, C═O), 144.87 (s, p-C_{Ts}), 133.35 (s, 132.98 (d, p-C_{Bz}), 130.94 (5, i-C_{Bz}), 129.97 (d, m-C_{Ts}), 129.72 (d, o-C_{Bz}), 128.58 (d, m-C_{Bz}), 128.13 (d, o-C_{Ts}), 74.21 (t, C-22), 72.03 (d, C-8), 52.44 (d), 51.52 (d), 41.82 (s, C-13), 39.30 (t), 35.00 (d), 30.57 (t), 26.56 (t), 22.54 (t), 21.85 (q, Me_{Ts}), 18.12 (t), 16.85 (q, C-21), 14.09 (q, C-18); MS (EI) m/z 470 (1, M⁺), 365 (33, M⁺-PhCO), 348 (64, M⁺-PhCOOH), 193 (52), 176 (71), 134 (72), 105 (100); exact mass calculated for C₂₇H₃₄O₅S (M⁺) 470.2127, found 470.2091.

### Preparation of (8S,20S)-Des-A,B-20-pentyl-pregnan-8-ol (7)

Magnesium turnings (625 mg, 26 mmol), 1-chloro-butane (1.5 mL, 1.3 g, 14 mmol) and iodine (2 crystals) were refluxed in anhydrous THF (13 mL) for 4 h. The solution of the formed Grignard reagent 6 was cooled to −78° C. and added dropwise via cannula to a solution of the tosylate 5 (170 mg, 0.36 mmol) in anhydrous THF (5 mL) at −78° C. Then 5 mL of the solution of Li₂CuCl₄ [prepared by dissolving dry LiCl (116 mg, 2.73 mmol) and dry CuCl₂ (184 mg, 1.36 mmol) in anhydrous THF (13 mL)] was added dropwise via cannula to the reaction mixture at −78° C. The cooling bath was removed and the mixture was stirred at room temperature for 20 h and then poured into 1M aq. H₂SO₄ solution (12 mL) containing ice (ca. 50 g). The mixture was extracted with methylene chloride (3×50 mL) and the combined organic layers were washed with saturated aq. NH₄Cl, saturated aq. NaHCO₃, dried (Na₂SO₄) and concentrated under reduced pressure. The residue was chromatographed on silica gel with hexane/ethyl acetate (96:4) to give alcohol 7 (61 mg, 67% yield) as a colorless oil: ): [α]_{D} +10.5 (c 1.4, CHCl₃); ¹H NMR (400 MHz, CDCl₃) δ 4.07 (1H, s, 8α-H), 1.98 (1H, m), 1.80 (3H, m), 0.92 (3H, s, 18-H₃), 0.88 (3H, d, J=7.0 Hz, 27-H₃), 0.81 (3H, d, J=6.6 Hz, 21-H₃); ¹³C NMR (100 MHz) δ 69.45 (d, C-8), 56.30 (d), 52.66 (d), 41.88 (s, C-13), 40.32 (t), 35.22 (t), 34.81 (d), 33.57 (t), 32.31 (t), 27.07 (t), 25.93 (t), 22.79 (t), 22.42 (t), 18.55 (q), 17.49 (1), 14.14 (q), 13.76 (q); MS (EI) m/z 252 (53, M⁺), 237 (37, M⁺-CH₃), 219 (9, M⁺-CH₃—H₂O), 163 (18, M⁺-C₅H₁₁—H₂O), 138 (68), 125 (69), 111 (100), 97 (51); exact mass calculated for C₁₇H₃₂O (M⁺) 2512.453, found 252.2450.

### Preparation of (20S)-Des-A,B-20-pentyl-pregnan-8-one (8)

Molecular sieves Å4 (150 mg) were added to a solution of 4-methylmorpholine oxide (20 mg, 0.2 mmol) in dichloromethane (0.7 mL). The mixture was stirred at room temperature for 15 min and tetrapropylammonium perruthenate (3 mg, 9 μmol) was added, followed by a solution of alcohol 7 (21 mg, 83 μmol) in dichloromethane (400+400 μL). The resulting suspension was stirred at room temperature for 1 h. The reaction mixture was filtered through a Waters silica Sep-Pak cartridge (2 g) that was further washed with dichloromethane. After removal of the solvent the ketone 8 (19 mg, 92% yield) was obtained as a colorless oil: [α]_{D} −37.7 (c 0.95, CHCl₃); MS (EI) m/z 250 (67, M⁺), 235 (67, M⁺-CH₃), 207 (76, M⁺-CH₃—H₂O), 151 (56, M⁺-C₅H₁₁—H₂O), 138 (46), 125 (100), 111 (93), 96 (68); exact mass calculated for C₁₇H₃₀O (M⁺) 250.2297, found 250.2304.

### Preparation of (20S)-2-Methylene-19,26-dinor-1α-hydroxyvitamin D₃ (11)

To a solution of phosphine oxide 9 (73 mg, 125 μmol) in anhydrous THF (500 μL) at −20° C. was slowly added PhLi (1.6 M in di-n-butylether, 100 μL, 160 μmol) under argon with stirring. The solution turned deep orange. After 30 min the mixture was cooled to −78° C. and a precooled (−78° C.) solution of ketone 8 (18 mg, 72 μmol) in anhydrous THF (200+100 μL) was slowly added. The mixture was stirred under argon at −78° C. for 4 h and at 0° C. for 18 h. Ethyl acetate was added, and the organic phase was washed with brine, dried (Na₂SO₄) and evaporated. The residue was dissolved in hexane and applied on a Waters silica Sep-Pak cartridge (2 g). The cartridge was washed with hexane and hexane/ethyl acetate (99.5:0.5) to give 19-norvitamin derivative 10 (31.6 mg, 71% yield). Then the Sep-Pak was washed with ethyl acetate to recover diphenylphosphine oxide 9 (33 mg). UV (in hexane) λₘₐₓ 262.5, 253.0, 245.0 nm; ¹H NMR (400 MHz, CDCl₃) δ 6.22 and 5.84 (each 1H, each d, J=11.1 Hz, 6- and 7-H), 4.98 and 4.93 (each 1H, each s, ═CH₂), 4.42 (2H, m, 1β- and 3α-H), 2.83 (1H, dm, J=11.6 Hz, 9β-H), 2.52 (1H, dd, J=13.3, 6.0 Hz, 10β-H), 2.47 (1H, dd, J=12.5, 4.4 Hz, 4α-H), 2.34 (1H, dd, J=13.3, 2.9 Hz, 10β-H), 2.18 (1H, dd, J=12.5, 8.4 Hz, 4β-H), 1.99 (2H, m), 0.900 (9H, s, Si-t-Bu), 0.892 (3H, t, 27-H₃), 0.868 (9H, s, Si-t-Bu), 0.84 (3H, d, J=6.5 Hz, 21-H₃), 0.544 (3H, s, 18-H₃), 0.083 (3H, s, SiMe), 0.069 (3H, s, 0.052 (9H, s, SiMe), 0.029 (3H, s, SiMe); ¹³C NMR (100 MHz) δ 152.98 (s, C-2), 141.27 (s, C-8), 132.67 (s, C-5), 122.43 (d, C-6), 116.08 (d, C-7), 106.25 (t, ═CH₂), 72.51 and 71.63 (each d, C-1 and C-3), 56.32 (d), 56.23 (d), 47.60 (t), 45.70 (s, C-13), 40.51 (t), 38.55 (t), 35.56 (d and t), 32.34 (t), 28.77 (t), 27.42 (t), 26.00 (t), 25.84 (q, SiCMe₃), 25.78 (q, SiCMe₃), 23.46 (t), 22.82 (t), 22.11 (t), 18.62 (q, C-21), 18.26 (s, SiCMe₃), 18.17 (s, SiCMe₃), 14.16 (q, C-27), 12.30 (q, C-18), −4.86 (q, 2×SiMe), −4.91 (q, SiMe), −5.10 (q, SiMe); exact mass calculated for C₃₈H₇₀O₂Si₂Na (MNa⁺) 637.4812, found 637.4837.

The protected vitamin 10 (31.5 mg, 51 mmol) was dissolved in THF (2 mL) and acetonitrile (2 mL). A solution of aq. 48% HF in acetonitrile (1:9 ratio, 2 mL) was added at 0° C. and the resulting mixture was stirred at room temperature for 6 h. Saturated aq. NaHCO₃ solution was added and the reaction mixture was extracted with ethyl acetate. The combined organic phases were washed with brine, dried (Na₂SO₄) and concentrated under reduced pressure. The residue was diluted with 2 mL of hexane/ethyl acetate (95:5) and applied on a Waters silica Sep-Pak cartridge (2 g). An elution with hexane/ethyl acetate (9:1) and later with ethyl acetate gave the crude product 11 (17 mg). The vitamin 11 was further purified by straight phase HPLC [9.4×250 mm Zorbax Sil column, 5 mL/min, hexane/2-propanol (9:1) solvent system, Rₜ=6.13 mm.] and later by reverse phase HPLC [9.4×250 mm Zorbax Eclipse XDB-C18 column, 3 mL/min, methanol/water (95:5) solvent system, Rₜ=14.69 min.] to give a colorless oil (14.2 mg, 72% yield): UV (in EtOH) λₘₐₓ 261.5, 252.5, 244.5 nm; ¹H NMR (500 MHz, CDCl₃) δ 6.35 and 5.88 (1H and 1H, each d, J=11.3 Hz, 6- and 7-H), 5.10 and 5.08 (each 1H, each s, ═CH₂), 4.46 (2H, m, 1β- and 3α-H), 2.84 (1H, dd, J=13.2, 4.5 Hz, 10β-H), 2.81 (1H, br d, J=12.6 Hz, 9β-H), 2.57 (1H, dd, J=13.3, 3.8 Hz, 4α-H), 2.32 (1H, dd, J=13.3, 6.2 Hz, 4β-H), 2.29 (1H, dd, J=13.2, 8.4 Hz, 10α-H), 1.98 (2H, m), 1.85 (1H, m), 0.88 (3H, t, J=7.1 Hz, 27-H₃), 0.83 (3H, d, J=6.5 Hz, 21-H₃), 0.543 (3H, s, 18-H₃); ¹³C NMR (125 MHz) δ 151.98 (s, C-2), 143.53 (s, C-8), 130.33 (s, C-5), 124.29 (d, C-6), 115.25 (d, C-7), 107.71 (t, ═CH₂), 71.81 and 70.67 (each d, C-1 and C-3), 56.36 (d), 56.22 (d), 45.83 (s, C-13), 45.78 (t), 40.34 (t), 38.14 (t), 35.53 (t), 35.48 (d), 32.30 (t), 28.97 (t), 27.29 (t), 25.95 (t), 23.50 (t), 22.77 (t), 22.16 (t), 18.57 (q, C-21), 14.12 (q, C-27), 12.32 (q, C-18); MS (EI) m/z 386 (84, M⁺), 368 (9, M⁺-H₂O), 353 (5, M⁺-H₂O—CH₃), 315 (17, M⁺-2H₂O—CH₃), 301 (47, M⁺-C₆H₁₃) 287 (53, M⁺C₇H₁₅), 269 (44), 251 (35), 233 (61), 177 (38), 147 (67), 135 (100); exact mass calculated for C₂₆H₄₂O₂ (M⁺) 386.3185, found 386.3174.

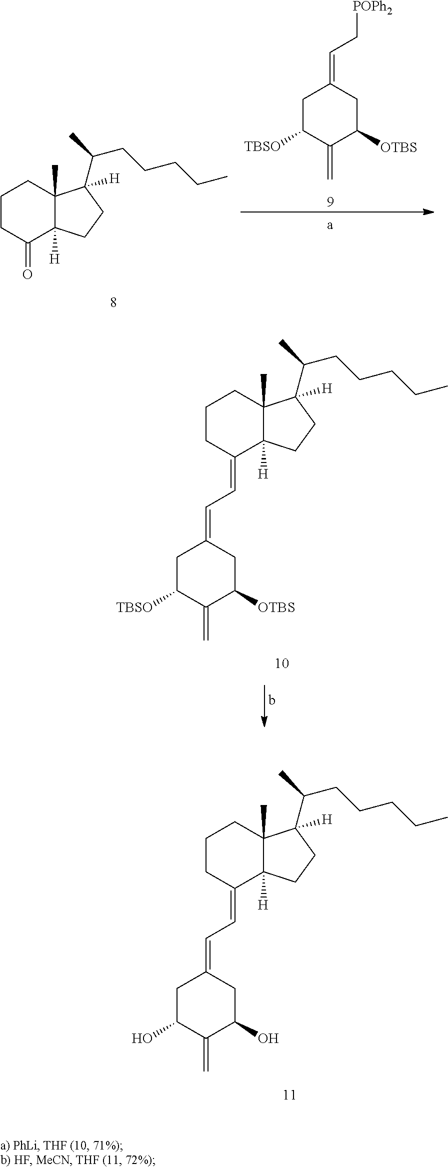

### Biological Activity

### Vitamin D Receptor Binding

### Test Material

Protein Source

Fun-length recombinant rat receptor was expressed in E. coli BL21(DE3) Codon Plus RIL cells and purified to homogeneity using two different column chromatography systems. The first system was a nickel affinity resin that utilizes the C-terminal histidine tag on this protein. The protein that was eluted from this resin was further purified using ion exchange chromatography (S-Sepharose Fast How). Aliquots of the purified protein were quick frozen in liquid nitrogen and stored at −80° C. until use. For use in binding assays, the protein was diluted in TEDK₅₀ (50 mM iris, 1.5 mM EDTA, pH 7.4, 5 mM DTT, 150 mM KCl) with 0.1% Chaps detergent. The receptor protein and ligand concentration was optimized such that no more than 20% of the added radiolabeled ligand was bound to the receptor.

Study Drugs

Unlabeled ligands were dissolved in ethanol and the concentrations determined using UV spectrophotometry (1,25(OH)₂D₃: molar extinction coefficient=18,200 and λₘₐₓ=265 nm; Analogs: molar extinction coefficient=42,000 and λₘₐₓ=252 nm). Radiolabeled ligand (³H-1,25(OH)₂D₃, ˜159 Ci/mmole) was added in ethanol at a final concentration of 1 nM.

Assay Conditions

Radiolabeled and unlabeled ligands were added to 100 met of the diluted protein at a final ethanol concentration of ≦10%, mixed and incubated overnight on ice to reach binding equilibrium. The following day, 100 mcl of hydroxylapatite slurry (50%) was added to each tube and mixed at 10-minute intervals for 30 minutes. The hydroxylapaptite was collected by centrifugation and then washed three times with Tris-EDTA buffer (50 mM Tris, 1.5 mM EDTA, pH 7.4) containing 0.5% Titron X-100. After the final wash, the pellets were transferred to scintillation vials containing 4 ml of Biosafe II scintillation cocktail, mixed and placed in a scintillation counter. Total binding was determined from the tubes containing only radiolabeled ligand.

HL-60 Differentiation

### Test Material

Study Drugs

The study drugs were dissolved in ethanol and the concentrations determined using UV spectrophotometry. Serial dilutions were prepared so that a range of drug concentrations could be tested without changing the final concentration of ethanol (≦10.2%) present in the cell cultures.

Cells

Human promyelocytic leukemia (HL60) cells were grown in RPMI-1640 medium containing 10% fetal bovine serum. The cells were incubated at 37° C. in the presence of 5% CO₂.

Assay Conditions

HL60 cells were plated at 1.2×10⁵ cells/ml. Eighteen hours after plating, cells in duplicate were treated with drug. Four days later, the cells were harvested and a nitro blue tetrazolium reduction assay was performed (Collins et al., 1979; J. Exp. Med. 149:969-974). The percentage of differentiated cells was determined by counting a total of 200 cells and recording the number that contained intracellular black-blue formazan deposits. Verification of differentiation to monocytic cells was determined by measuring phagocytic activity (data not shown).

### In Vitro Transcription Assay

Transcription activity was measured in ROS 17/2.8 (bone) cells that were stably transfected with a 24-hydroxylase (24OHase) gene promoter upstream of a luciferase reporter gene (Arbour et al., 1998). Cells were given a range of doses. Sixteen hours after dosing the cells were harvested and luciferase activities were measured using a luminometer. RLU=relative luciferase units.

### Intestinal Calcium Transport and Bone Calcium Mobilization

Male, weanling Sprague-Dawley rats were placed on Diet 11 (0.47% Ca) diet+AEK oil for one week followed by Diet 11 (0.02% Ca)+AEK oil for 3 weeks. The rats were then switched to a diet containing 0.47% Ca for one week followed by two weeks on a diet containing 0.02% Ca. Dose administration began during the last week on 0.02% calcium diet. Four consecutive intraperitoneal doses were given approximately 24 hours apart. Twenty-four hours after the last dose, blood was collected from the severed neck and the concentration of serum calcium determined as a measure of bone calcium mobilization. The first 10 cm of the intestine was also collected for intestinal calcium transport analysis using the everted gut sac method.

### Biological Activity Results

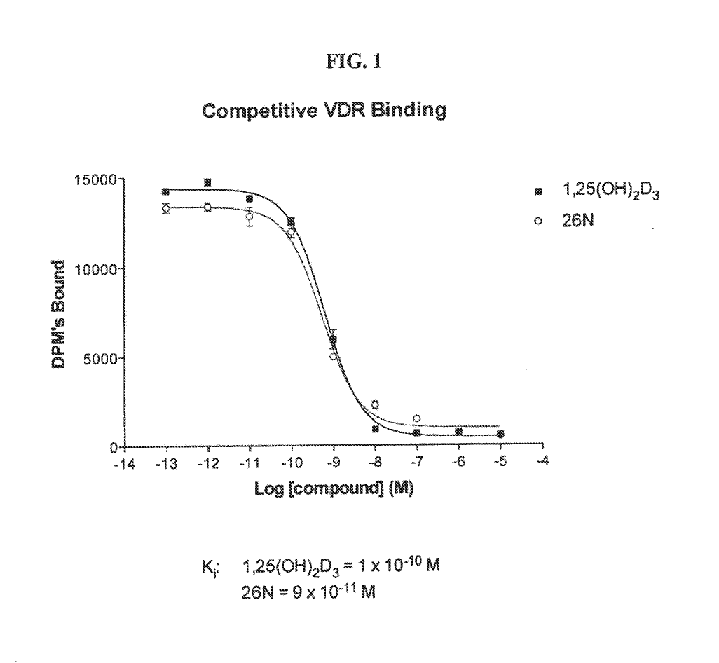

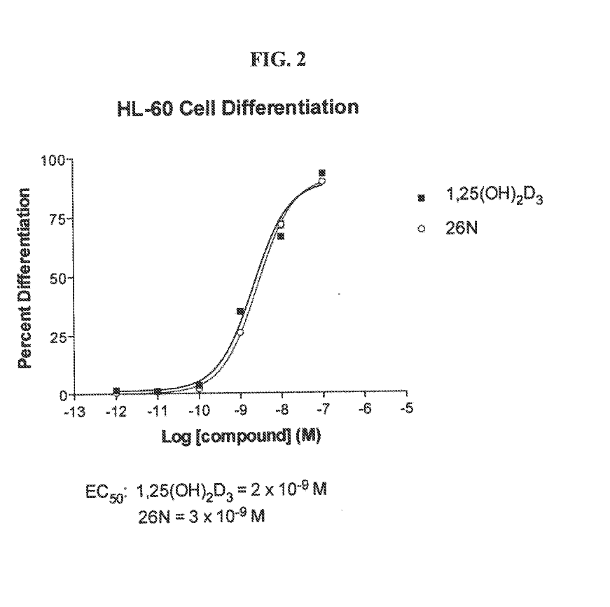

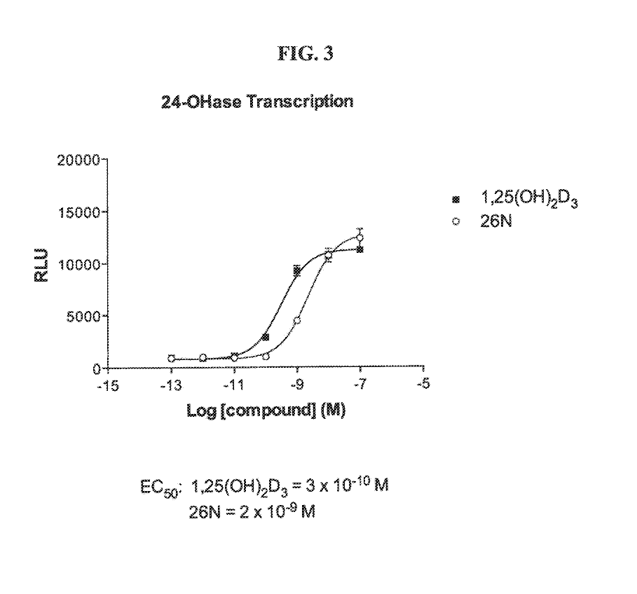

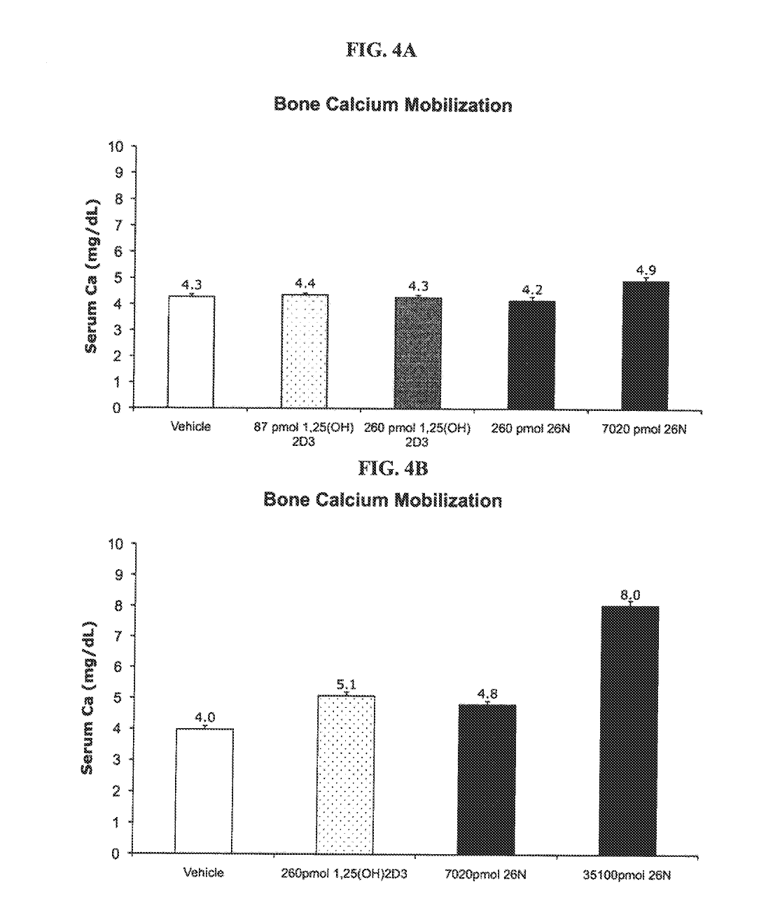

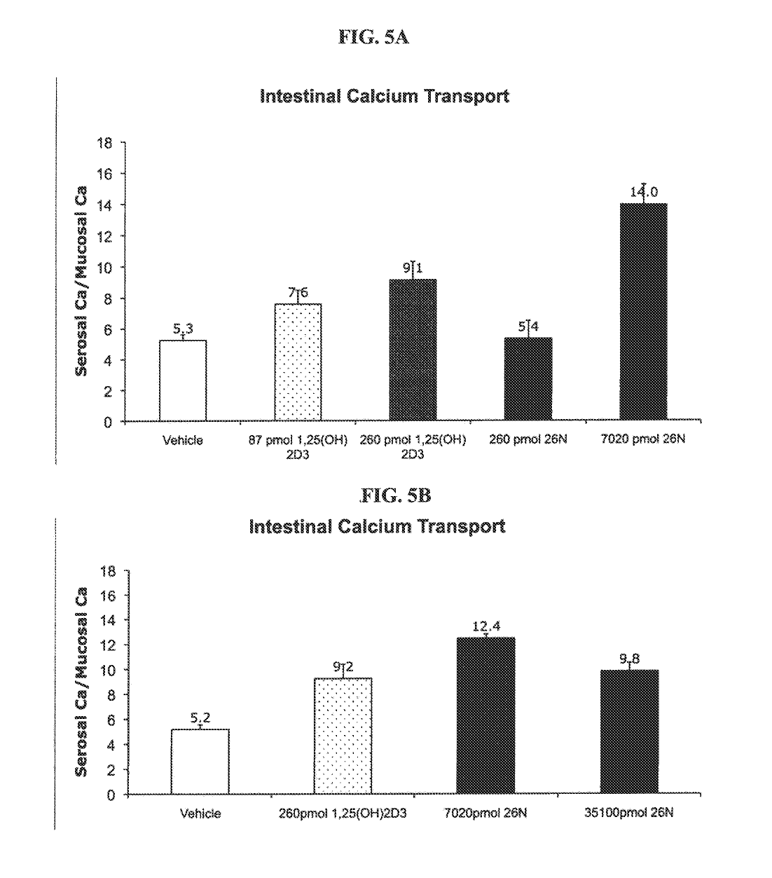

The biological activity of 2-methylene-19,26-nor-(20S)-1α-hydroxyvitamin D3 was assayed using the methods described above. Results of the assays are presented in FIGS. 1-5. 2-Methylene-19,26-nor-(20S)-1α-hydroxyvitamin D3 is approximately equally effective as 1,25-(OH)₂D₃ in binding to the recombinant vitamin D receptor as shown in FIG. 1. It also induces the differentiation of HL-60 cells in culture (FIG. 2) with the same potency as 1,25-(OH)₂D₃. However, it is about 10 times less potent in stimulating 24-OHase gene expression in bone cells than 1,25-(OH)₂D (FIG. 3). As shown in FIGS. 4A, 4B, 5A and 5B, the calcemic activity of 2-methylene-19,26-nor-(20S)-1α-hydroxyvitamin D3 is also very low.

Preliminary in vivo tests of bone calcium mobilization activity demonstrated that that 2-methylene-19,26-nor-(20S)-1α-hydroxyvitamin D₃ displayed essentially no calcemic activity at a 260 pmol dose (FIG. 4A). Further in vivo testing demonstrated that 2-methylene-19,26-nor-(20S)-1α-hydroxyvitamin D3 is nearly 30 times less active than 1,25-(OH)₂D₃ on bone calcium mobilization as shown at the dose of 7020 pmol (FIG. 4B), and noticeably less active than 1,25-(OH)₂D₃ in causing intestinal calcium transport (FIGS. 5A, 5B).

The low calcemic activity measured for 2-methylene-19,26-nor-(20S)-1α-hydroxyvitamin D3 is surprising in view of the calcemic activity produced by 2-methylene-19-nor-(20S)-1α-hydroxyvitamin D3. Table 1 lists the calcemic activity of 2-methylene-19,26-nor-(20S)-1α-hydroxyvitamin D3 (referred to as “26N”) to that of 2-methylene-19-nor-(20S)-1α-hydroxyvitamin D3 (referred to as “26Me”) as reported by Grzywacz et al. Arch. Biochem. Biophys., 460, 274 (2007), which is hereby incorporated by reference in its entirety as if fully set forth herein. For the purposes of the comparison, the net bone calcium mobilization activity and intestinal calcium transport activity are presented alter subtraction of the corresponding activity observed for the vehicle alone.

TABLE 1

| | | | Intestinal calcium |
|---|---|---|---|
| | | Bone calcium | transport (change from |
| | Dose level | mobilization (change | vehicle, serosal/mucosal |
| | (pmol) | from vehicle, mg/dL) | ratio) |
| 26N | 260 | −0.1 | 0.1 |
| | 7020 | 0.6, 0.8 | 8.7, 7.2 |
| | 35100 | 4.0 | 4.6 |
| 26Me | 87 | 2.0 | 6.6 |
| | 260 | 4.0 | 5.3 |
| | 780 | 5.3 | 2.3 |
| | 2340 | 5.6 | 5.4 |

Structurally, 2-methylene-19-nor-(20S)-1α-hydroxyvitamin D3 differs from 2-methylene-19,26-nor-(20S)-1α-hydroxyvitamin D3 in that the former possesses a 26-methyl group. Despite this small structural difference, the two compounds exhibit remarkably different biological properties with respect to calcemic activity. As shown in Table 1, 2-methylene-19,26-nor-(20S)-1α-hydroxyvitamin D3 exhibits essentially no calcemic activity with respect to either bone calcium mobilization or intestinal calcium transport at a 260 pmol dose. Even at 7020 pmol, 26N shows little bone calcium mobilization activity. In contrast, 2-methylene-19-nor-(20S)-1α-hydroxyvitamin D3 exhibits significant activity in both bone calcium mobilization and intestinal calcium transport at the same 260 pmol dose and at 2340 pmol shows nearly 10 times the bone calcemic activity of 26N at the higher dose of 7020 pmol.

The biological properties displayed by 2-methylene-19-nor-(20S)-1α-hydroxyvitamin D3 illustrate that this compound should be very useful in the treatment of diseases where a rise in serum calcium is not desirable. Thus, this compound should find utility in the treatment of secondary hyperparathyroidism of patients suffering from chronic kidney failure because it is undesirable to elevate serum calcium above normal in these patients for fear of calcification of heart, aorta and other vital organs while it suppresses parathyroid gland proliferation and transcription of the preproparathyroid gene. Likewise, this compound should be useful in the treatment of malignancy such as breast, colorectal and prostate cancers, or in the treatment of autoimmune diseases such as multiple sclerosis, lupus; rheumatoid arthritis, type 1 diabetes, and inflammatory bowel disease. It should also be useful in preventing transplant rejection.

It is to be understood that the present technology is not limited to the embodiments set forth herein for illustration, but embraces all such forms thereof as come within the scope of the following claims.

What is claimed is:

1. A compound having the formula I 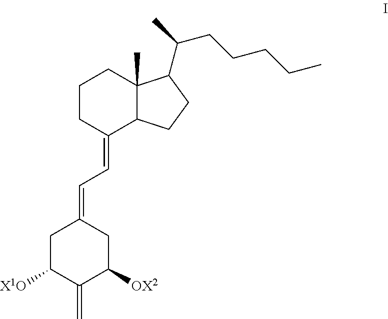
wherein X¹ and X² are independently selected from H and hydroxy protecting groups.

2. The compound of claim 1, wherein X¹ and X² are both hydroxy protecting groups.

3. The compound of claim 2, wherein X¹ and X² are both t-butyldimethylsilyl groups.

4. The compound of claim 1, wherein X¹ and X² are both H and the compound has the formula IA

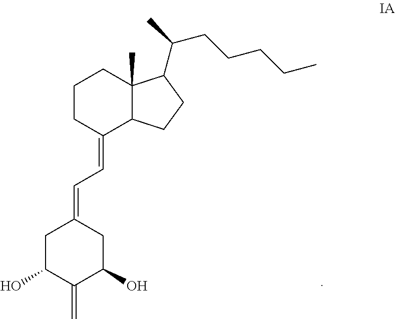

5. A pharmaceutical composition, comprising an effective amount of the compound of claim 4 and a pharmaceutically acceptable carrier.

6. The pharmaceutical composition of claim 5 wherein the effective amount comprises from about 0.01 μg to about 1 mg of the compound per gram of the composition.

7. The pharmaceutical composition of claim 5 wherein the effective amount comprises from about 0.1 μg to about 500 μg of the compound per gram of the composition.

8. A method of treating a subject suffering from a biological condition, comprising administering an effective amount of the compound of claim 4 to the subject, wherein the biological condition is selected from psoriasis; leukemia; colon cancer; breast cancer; prostate cancer; multiple sclerosis; lupus; diabetes mellitus; host versus graft reaction; rejection of organ transplants; an inflammatory disease selected from rheumatoid arthritis, asthma, or inflammatory bowel diseases; a skin condition selected from wrinkles, lack of adequate skin firmness, lack of adequate dermal hydration, or insufficient sebum secretion; renal osteodystrophy; or osteoporosis.

9. The method of claim 8, wherein the biological condition is psoriasis.

10. The method of claim 8, wherein the biological condition is selected from leukemia, colon cancer, breast cancer, or prostate cancer.

11. The method of claim 8, wherein the biological condition is selected from multiple sclerosis, lupus, diabetes mellitus host versus graft reaction, or rejection of organ transplants.

12. The method of claim 8, wherein the biological condition is selected from rheumatoid arthritis, asthma, or inflammatory bowel diseases selected from celiac disease, ulcerative colitis and Crohn's disease.

13. The method of claim 8, wherein the biological condition is selected from wrinkles, lack of adequate skin firmness, lack of adequate dermal hydration, or insufficient sebum secretion.

14. The method of claim 8, wherein the compound is administered orally to the subject.

15. The method of claim 8, wherein the compound is administered parenterally to the subject.

16. The method of claim 8, wherein the compound is administered transdermally to the subject.

17. The method of claim 8, wherein the compound is administered topically to the subject.

18. The method of claim 8, wherein the compound is administered in a dosage of from about 0.01 μg per day to about 1 mg per day.

19. The compound of claim 1, wherein X¹ and X² are both H and the compound has the formula IB [chemical structure]
